(19) United States
(12) Patent Application Publication
(10) Pub. No.: US 2016/0367617 A1
(43) Pub. Date: Dec. 22, 2016

(54) HERBAL PRODUCT FOR ITS ADMINISTRATION TO DIABETIC PEOPLE AND PROCESS TO OBTAIN IT

(71) Applicants: Juan Carlos AGREDA NAVAJAS, Vitoria (ES); Fidel MARTIN PINTO, Vitoria (ES)

(72) Inventors: Juan Carlos AGREDA NAVAJAS, Vitoria (ES); Fidel MARTIN PINTO, Vitoria (ES); Efren William BELO MALUENDAS, Vitoria (ES)

(21) Appl. No.: 15/164,392

(22) Filed: May 25, 2016

Related U.S. Application Data
(62) Division of application No. 12/114,986, filed on May 5, 2008, now abandoned.
Application No. 12/114,986 is a continuation-in-part of application No. 10/512,209, filed on Oct. 22, 2004, now abandoned.

(30) Foreign Application Priority Data
Apr. 23, 2002 (ES) P 200200943

(51) Int. Cl. A61K 36/61; A61K 36/48

(52) U.S. Cl. CPC A61K 36/61; A61K 36/48

(57) ABSTRACT

Herbal product, for its administration to diabetic people and process to obtain it. The product is made up of the mixture of two plants, one of them belonging to the Myrtaceae family, preferably of the Syzygium species and the other to the leguminosae caesalpinoideae family and preferably of the Bauhinia variety. Its production process is developed by the harvesting and separation of the Syzygium seeds and the Bauhinia leaves; their cleaning by pulverizing; their drying till reaching 9% humidity and their grinding, sterilization and homogeneous mixing in the established amounts.

The object of the present invention is a herbal product developed and appropriate for the administration to diabetic people and more specifically to people who suffer from diabetes type II, not dependent from insulin.

The formulation of this herbal product is made up of the mixture of two plants, one of them belonging to the Myrtaceae and the other to the family of the leguminosae caesalpinoideae. Specifically, the first of them is a Syzygium and the second a Bauhinia. More specifically, the Syzygium plant contemplated as within the scope of the invention is Syzygium cumini that is synonymous to Eugenia cumini, Euginia jambolana, Myrtus cumini and Syzygium jambolanum, which all have other common names such as those delineated on the U.S. Department of Agriculture Web site www.ars-grin.gov/cgi-bin/npgs/html/taxon.pl?36128; and the Bauhinia plant contemplate as within the scope of the invention is Bauhinia forficata with its subspecie forficata or subspecie pruinosa, which is also synonymous with Bauhinia Candicans as also delineated in U.S. Department of Agriculture Web site www.ars-grin.gov/cgi-bin/npgs/html/taxon.pl?6566.

This product has a hypoglycaemic activity. Its mimetic activity with human insulin allows to reduce the glycose levels in patients with diabetes type II; in an unedited way in pharmacology of herbal formulations.

On the other hand and as they are products obtained from the mixture of two natural plants, the collateral effects on the patients are low or almost nonexistent, which supposes a great advantage as compared to the traditional chemical formulae.

According to the present invention a herbal product is tried to obtain, for its administration to diabetic patients, and more specifically to those suffering from type II, not insulin dependent diabetes.

### Formulation

The qualitative formulation of this product is the one made up of the mixture of two plants, one of them belonging to the Myrtaceae and the other to the family of the leguminosae-caesalpinoideae.

Specifically, the plant belonging to the Myrtaceae is a Syzygium and more specifically in a preferable but not limitative way the synonymous species of Syzygium cumini discussed above as selected varieties.

The plant of the leguminosae caesalpinoideae family is a Bauhinia forficata and its related species and subspecies discussed above.

For purposes of generically describing the above plants contemplated as within the scope of the invention, the following description as well as the claims appended hereto define the plants as Syzynium or Syzygium cumini and Bauhinia or Bauhinia forficata, with any above mentioned synonymous subspecie and common names being considered equivalents thereof.

The quantitative formulae is determined by a percentage by weight included between 50% and 95% of the Syzygium and a percentage by weight included between 5% and 50% of Bauhinia.

Within these percentages by weight satisfactory results have been reached with a percentage by weight of 85% of Syzygium and a percentage by weight of 15% of Bauhinia.

Particularly samples have been prepared of the product in which the weight of the plants was 450 mg. In this case, the amount of Syzygium is 382.5 mg and the amount of Bauhinia is 67.5 mg.

The presentation of the product can be any of the ones allowed by the pharmacopean techniques but preferably it shall be presented in capsules.

### Production Process

The process is started with the gathering or harvesting of the plants, after which the Syzygium seeds are cleaned as well as the Bauhinia leaves.

Both the seeds of Syzygium and the leaves of Bauhinia are pulverized to 60 mesh and afterwards dried till reaching 9% humidity.

After a quality control process, the grinding phase is started, in which the seeds and the leaves are ground, after which they are sterilized.

In the following phase both ground plants are mixed till reaching a perfect homogenization, after which they are capsulized after passing the corresponding quality controls. Finally the finished product is packed.

### Dosage

As a possible administration dose, satisfactory results have been reached with the following dosage:
A first attacking dose, according to which 4 capsules of 450 mg should be taken, each of them daily for a period of one week.
A maintenance dose of two capsules of 450 mg, each of them daily during the remaining 51 weeks, to complete, between the attacking dose and the maintenance one a period of one year.

It is convenient that the capsule ingestion is carried out twice a day; so that during the attacking dose two capsules shall be taken before breakfast in the morning and two other ones before dinner.

A capsule shall be taken before breakfast and another one before dinner for the maintenance dose.

1-11. (canceled)

12. A method of reducing blood glucose levels in an individual, said method comprising administering to the individual a synergistic dry composition comprising dry pulverized Syzygium cumini seeds and dry pulverized Bauhinia forficata leaves, wherein the administration of the synergistic dry composition reduces blood glucose level in the individual.

13. The method according to claim 12, wherein the individual suffers from diabetes.

14. The method according to claim 13, wherein the diabetes is type II diabetes.

15. The method according to claim 12, wherein the percentage by weight of dry pulverized Syzygium cumini seeds is between 50% and 95% and the percentage by weight of dry pulverized Bauhinia forficata leaves is between 5% and 50%.

16. The method according to claim 15, wherein the percentage by weight of dry pulverized Syzygium cumini seeds is about 85% and the percentage by weight of dry pulverized Bauhinia forficata leaves is about 15%.

17. The method according to claim 12, wherein the synergistic dry composition comprising dry pulverized Syzygium cumini seeds and dry pulverized Bauhinia forficata leaves is in capsule form.

18. The method according to claim 17, wherein the capsule comprises about 450 mg of the synergistic dry composition comprising dry pulverized Syzygium cumini seeds and dry pulverized Bauhinia forficata leaves.

19. The method according to claim 12, wherein the pulverized Syzygium cumini seeds and pulverized Bauhinia forficata leaves are pulverized to 60 mesh size.

20. The method according to claim 12, wherein the synergistic dry composition comprising dry pulverized Syzygium cumini seeds and dry pulverized Bauhinia forficata leaves is administered daily.

21. The method according to claim 12, wherein the synergistic dry composition comprising dry pulverized Syzygium cumini seeds and dry pulverized Bauhinia forficata leaves is administered twice a day.

22. The method according to claim 12, wherein the synergistic dry composition comprising dry pulverized Syzygium cumini seeds and dry pulverized Bauhinia forficata leaves is administered once or twice a day.

23. The method according to claim 22, wherein the synergistic dry composition comprising dry pulverized Syzygium cumini seeds and dry pulverized Bauhinia forficata leaves is administered in a dose comprising one or two capsules.

24. A synergistic dry composition comprising dry pulverized Syzygium cumini seeds and dry pulverized Bauhinia forficata leaves, wherein the administration of the synergistic dry composition to an individual with diabetes reduces blood glucose level in the individual.

25. The synergistic dry composition according to claim 24, wherein the diabetes is type II diabetes.

26. The synergistic dry composition according to claim 24, wherein the percentage by weight of dry pulverized Syzygium cumini seeds is between 50% and 95% and the percentage by weight of dry pulverized Bauhinia forficata leaves is between 5% and 50%.

27. The synergistic dry composition according to claim 26, wherein the percentage by weight of dry pulverized Syzygium cumini seeds is about 85% and the percentage by weight of dry pulverized Bauhinia forficata leaves is about 15%.

28. The synergistic dry composition according to claim 27, wherein the synergistic dry composition comprising dry pulverized Syzygium cumini seeds and dry pulverized Bauhinia forficata leaves is in capsule form.

29. The synergistic dry composition according to claim 28, wherein the capsule comprises about 450 mg of the synergistic dry composition comprising dry pulverized Syzygium cumini seeds and dry pulverized Bauhinia forficata leaves.

30. The synergistic dry composition according to claim 24, wherein the pulverized Syzygium cumini seeds and pulverized Bauhinia forficata leaves are pulverized to 60 mesh size.

31. A method of manufacturing a synergistic dry composition to reduce blood glucose level in an individual comprising combining dry pulverized Syzygium cumini seeds and dry pulverized Bauhinia forficata leaves.
